(19) United States
(12) Patent Application Publication
(10) Pub. No.: US 2003/0203921 A1
(43) Pub. Date: Oct. 30, 2003

(54) GABA analogs and an antiviral agent to treat shingles

(76) Inventors: Leslie Magnus, Livingston, NJ (US); Catherine Segal, Chester, NJ (US)

Correspondence Address: Mehdi Ganjeizadeh, Warner-Lambert Company LLC, 2800 Plymouth Road, Ann Arbor, MI, 48105, US

(21) Appl. No.: 10/405,314

(22) Filed: Apr. 2, 2003

Related U.S. Application Data
(63) Continuation of application No. 09/743,392, filed on Jan. 9, 2001, now abandoned.
Application No. 09/743,392 is a 371 of international application No. PCT/US99/13947, filed on Jun. 18, 1999.
(60) Provisional application No. 60/092,171, filed on Jul. 9, 1998.

(51) Int. Cl. A61K 31/522; A61K 31/513; A61K 31/198

(52) U.S. Cl. 514/263.31; 514/269; 514/561; 514/530

(57) ABSTRACT

The instant invention is a method of using certain analogs of glutamic acid and gamma-aminobutyric acid in combination with an anti-viral agent to treat shingles.

### BACKGROUND OF THE INVENTION

1. Field of the Invention

The present invention relates to the use of analogs of glutamic acid and gamma-aminobutyric acid (GABA) in combination with an antiviral agent, for the treatment of shingles.

2. Description of Related Art

The GABA analogs used in the present invention are known agents useful in antiseizure therapy for central nervous system disorders such as epilepsy, Huntington's chorea, cerebral ischemia, Parkinson's disease, tardive dyskinesia, and spasticity. It has also been suggested that the compounds can be used as antidepressants, anxiolytics, and antipsychotics. See WO 92/09560 (U.S. Ser. No. 618,692 filed Nov. 27, 1990) and WP 93/23383 (U.S. Ser. No. 886,080 filed May 20, 1992).

WO 97/33858 teaches that compounds related to gabapentin are useful or treating epilespy, faintness attacks, hypokinesia, cranial disorders, neurodegenerative disorders, depression, anxiety, panic, pain, and neuropathological disorders. WO 97/33858 does not specify what forms of pain are treated.

Additionally, the GABA analogs compounds of the present invention are known for treatment of neuropathic pain. For example, see Rosner H; Rubin L; Kestenbaum A., Gabapentin adjunctive therapy in neuropathic pain states. Clin J Pain, 1996 Mar, 12:1, 56-8; Segal A Z; Rordorf G., Gabapentin as a novel treatment for postherpetic neuralgia. Neurology, 1996 Apr, 46:4, 1175-6; Wetzel C H; Connelly J F., Use of gabapentin in pain management. Ann Pharmacother, 1997 Sep, 31:9, 1082-3; Zapp J J., Postpoliomyelitis pain treated with gabapentin [letter]. Am Fam Physician, 1996 Jun, 53:8, 2442, 2445; Cheville A, et al., Neuropathic pain in radiation myelopathy:a case report. Program book, American Pain Society (14th Annual Scientific Meeting). Abstract #95823, p. A-1 15; Sist T; Filadora V; Miner M; Lema M., Gabapentin for idiopathic trigeminal neuralgia: report of two cases. Neurology, 1997 May, 48:5, 1467; Waldman S D, Tutorial 28: Evaluation and Treatment of Trigeminal Neuralgia. Pain Digest (1997) 7:21-24; Mellick L B; Mellick G A., Successful treatment of reflex sympathetic dystrophy with gabapentin [letter]. Am J Emerg Med, 1995 Jan, 13:1, 96; Mellick G A; Seng M I., The use of gabapentin in the treatment of reflex sympathetic dystrophy and a phobic disorder. Am J Pain Manage 1995; 5:7-9; Mellick G A; Mellicy L B; Mellick L B., Gabapentin in the management of reflex sympathetic dystrophy [letter]. J Pain Symptom Manage, 1995 May, 10:4, 265-6; Mellick G A; Mellick L B., Reflex sympathetic dystrophy treated with gabapentin. Arch Phys Med Rehabil, 1997 Jan, 78:1, 98-105 and Mackin G A., Medical and pharmacologic management of upper extremity neuropathic pain syndromes. J Hand Ther, 1997 Apr-Jun, 10:2, 96-109.

U.S. Pat. No. 5,589,180 teaches a plaster composition for treating pain from herpes zoster or post perpetic neuralgia comprising an adhesive containing 2-10% by weight lidocaine, at least one of propylene glycol and clycerin as a co-solvent and a covering.

Antiviral compounds are known to treat herpes. Thes compounds include acyclovir, famciclovir, valacylovir, peniclovir and mixtures thereof. These antiviral compounds interfere with the enxyme thymidine kinase that is needed to for the replication of the herpes virus.

### SUMMARY OF THE INVENTION

This invention provides a method for treating shingles comprising administering to a subject suffering from shingles an effective amount of a GABA analog and an antiviral agent. A preferred embodiment utilizes a cyclic amino acid compound of Formula I

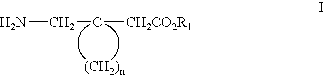

wherein R₁ is hydrogen or lower alkyl and n is an integer of from 4 to 6, and the pharmaceutically acceptable salts thereof. An especially preferred embodiment utilizes a compound of Formula I where R₁ is hydrogen and n is 4, which compound is 1-(aminomethyl)-cyclohexane acetic acid, known generically as gabapentin.

In another embodiment, the invention includes treating shingles with a compound of Formula II and an antiviral agent.

Formula II

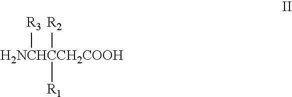

or a pharmaceutically acceptable salt thereof wherein

R₁ is a straight or branched alkyl of from 1 to 6 carbon atoms, phenyl, or cycloalkyl of from 3 to 6 carbon atoms;

R₂ is hydrogen or methyl; and

R₃ is hydrogen, methyl, or carboxyl.

Preferred compounds of the invention are those wherein R₃ and R₂ are hydrogen, and R₁ is —(CH₂)₀₋₂-i C₄H₉ as an (R), (S), or (R,S) isomer.

The more preferred compounds of Formula II invention are (S)-3-(aminomethyl)-5-methylhexanoic acid and 3-aminomethyl-5-methyl-hexanoic acid, now known generically as pregabalin.

### DETAILED DESCRIPTION OF THE PREFERRED EMBODIMENTS

The method of this invention utilizes any GABA analog. A GABA analog is any compound derived from or based upon gamma-aminobutyric acid. The compounds are readily available, either commercially, or by synthetic methodology well-known to those skilled in the art of organic chemistry. The preferred GABA analogs to be utilized in the method of this invention are cyclic amino acids of Formula I. These are described in U.S. Pat. No. 4,024,175, which is incorporated herein by reference. Another preferred method utilizes the GABA analogs of Formula II, and these are described in U.S. Pat. No. 5,563,175 which is incorporated herein by reference.

All that is required to practice the method of this invention is to administer a GABA analog in an amount that is effective to treat shingles. Such amounts will generally be from about 1 to about 300 mg per kg of subject body weight. Typical doses will be from about 10 to about 5000 mg per day for an adult subject of normal weight. It is expected that common doses that might be administered could be from 100 mg three times a day up to 600 mg four times a day. Commercially available capsules of 100 mg, 300 mg, and 400 mg of gabapentin can be administered. Alternate forms include liquids and film-coated tablets.

If a compound of Formula II, such as pregabalin is used, the dosage level is one sixth that of gabapentin. The dosage range for pregabalin is from about 0.15 mg to about 50 mg per kg per day of subject body weight. Typical dosages for pregabalin will be from about 1.6 mg to about 840 mg per day with individual dosages ranging from abut 0.15 mg to about 65 mg per dose.

The compounds of the present invention may form pharmaceutically acceptable salts with both organic and inorganic acids or bases. For example, the acid addition salts of the basic compounds are prepared either by dissolving the free base in aqueous or aqueous alcohol solution or other suitable solvents containing the appropriate acid and isolating the salt by evaporating the solution. Examples of pharmaceutically acceptable salts are hydrochlorides, hydrobromides, hydrosulfates, etc. as well as sodium, potassium, and magnesium, etc. salts.

The compounds of the Formula II can contain one or several asymmetric carbon atoms. The invention includes the individual diastereomers or enantiomers, and the mixtures thereof. The individual diastereomers or enantiomers may be prepared or isolated by methods already well-known in the art.

Pharmaceutical compositions of the compound of the present invention or its salts are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Some examples of suitable pharmaceutical carriers, including pharmaceutical diluents, are gelatin capsules; sugars such as lactose and sucrose; starches such as corn starch and potato starch, cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations. The compositions of the invention can also contain other components such as coloring agents, flavoring agents, and/or preservatives. These materials, if present, are usually used in relatively small amounts. The compositions can, if desired, also contain other therapeutic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present.

Routes of administration of the subject compound or its salts are oral or parenteral. For example, a useful intravenous dose is between 5 and 50 mg and a useful oral dosage is between 20 and 800 mg. The dosage is within the dosing range used in treatment of pain or as would be with the needs of the patient as described by the physician.

A unit dosage form of the GABA analog to be used in this invention may also comprise other compounds useful in the treatment of pain.

The advantages of using the compounds of Formula I and II, especially gabapentin and pregabalin, in the instant invention include the relatively nontoxic nature of the compounds, the ease of preparation, the fact that the compounds are well-tolerated, and the ease of IV administration of the drugs. Gabapentin has few interactions with major classes of drugs since it is not metabolized in the liver, but rather excreted unchanged from the body. Further, the drugs are not metabolized in the body. The subjects treated with the method of the present invention are mammals, including humans.

The antiviral compositions used in the present invention reduce the viral load thereby reducing the number of days of suffering. GABA analogs have no direct impact on the viral load. The GABA analogs work to diminish the pain signals begin transmitted from the peripheral nerves to the brain. The combination of actions improve control and pain relief during a shingles infection.

We claim:

1. A method for treating sinus headache or sinus pain, comprising administering a pharmaceutical composition comprising:
(a) an analgesically effective amount of a GABA analog; and
(b) an effective amount of a anti-viral agent.

2. The method according to claim 1, wherein the GABA analog is the compound according to Formula I: 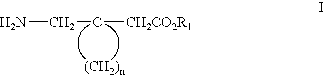
wherein R₁ is hydrogen or lower alkyl and n is an integer of from 4 to 6, and the pharmaceutically acceptable salts thereof.

3. The method according to claim 2, wherein Formula I comprises gabapentin.

4. The method according to claim 1, wherein the anti-viral agent is selected from the group consisting of acyclovir, famciclovir, valacylovir, peniclovir and mixtures thereof.

5. The method according to claim 2, comprising from about 10 mg to about 400 mg of Formula I.

6. The method according to claim 3, comprising from about 10 mg to about 400 mg of gabapentin.

7. The method according to claim 3, comprising from about 10 mg to about 400 mg of gabapentin and from about 60 mg to about 200 mg of anti-viral agent.

8. The method according to claim 1, wherein the GABA analog is a compound according to Formula II: 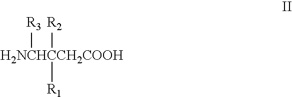
or a pharmaceutically acceptable salt thereof wherein
R₁ is a straight or branched alkyl of from 1 to 6 carbon atoms, phenyl, or cycloalkyl of from 3 to 6 carbon atoms;
R₂ is hydrogen or methyl; and
R₃ is hydrogen, methyl, or carboxyl.

9. The method according to claim 8, wherein Formula II comprises pregabalin.

10. The method according to claim 8, comprising from about 0.15 mg to about 65 mg of Formula II.

11. The method according to claim 9, comprising from about 0.15 mg to about 65 mg of pregabalin.

12. A composition for eliciting an enhanced analgesic response in a mammal comprising:
(a) an analgesically effective amount of a GABA analog; and
(b) an effective amount of a anti-viral agent.

13. The composition according to claim 12, wherein the GABA analog the compound according to Formula I: 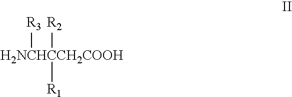
wherein R₁ is hydrogen or lower alkyl and n is an integer of from 4 to 6, and the pharmaceutically acceptable salts thereof.

14. The composition method according to claim 13, wherein Formula I comprises gabapentin.

15. The composition according to claim 13, comprising from about 10 mg to about 400 mg of Formula I.

16. The composition according to claim 14, comprising from about 10 mg to about 400 mg of gabapentin.

17. The composition according to claim 12, wherein the GABA analog is a compound according to Formula II: 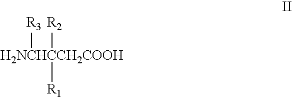
or a pharmaceutically acceptable salt thereof wherein
R₁ is a straight or branched alkyl of from 1 to 6 carbon atoms, phenyl, or cycloalkyl of from 3 to 6 carbon atoms;
R₂ is hydrogen or methyl; and
R₃ is hydrogen, methyl, or carboxyl.

18. The composition according to claim 17, wherein Formula II comprises pregabalin.

19. The composition according to claim 17, comprising from about 0.15 mg to about 65 mg of Formula II.

20. The composition according to claim 19, comprising from about 0.15 mg to about 65 mg of pregabalin.
